(19) United States
(12) Patent Application Publication
(10) Pub. No.: US 2006/0063154 A1
(43) Pub. Date: Mar. 23, 2006

(54) Methods and materials for reducing production of aberrant products during rna synthesis

(76) Inventors: William T McAllister, Perth Amboy, NY (US); Alexander Kukarin, Forest Hills, NY (US)

Correspondence Address: HOFFMAN WARNICK & D'ALESSANDRO, LLC, 75 STATE STREET, 14TH FL, ALBANY, NY, 12207, US

(21) Appl. No.: 10/537,956

(22) PCT Filed: Dec. 10, 2003
(86) PCT No.: PCT/US03/39087

§ 371 Date: Jun. 8, 2005

Related U.S. Application Data
(60) Provisional application No. 60/432,432, filed on Dec. 11, 2002.

(51) Int. Cl. C12Q 1/68; C12N 9/22

(52) U.S. Cl. 435/6; 435/199

(57) ABSTRACT

Improved materials and methods for reducing the production of aberrant products during RNA synthesis and protein production are provided.

### CROSS REFERENCE TO RELATED APPLICATIONS

This application claims the benefit of co-pending P.C.T. Application No. PCT/US2003/39087, filed on Dec. 10, 2003, and U.S. Provisional Application Ser. No. 60/432,432, filed on Dec. 11, 2002, and hereby incorporated herein by reference.

### STATEMENT OF GOVERNMENT INTEREST

This work was supported by a grant (Project 10009175, Award 006328, ID number GM38147) from the National Institutes of Health.

### FIELD OF THE INVENTION

This invention relates to improved materials and methods for reducing the production of aberrant products during RNA synthesis and protein production.

### BACKGROUND OF THE INVENTION

RNA polymerases are ubiquitous in nature and used extensively in the biotechnology industry. They are employed in nucleic acid amplification reactions with reverse transcriptase and RnaseH to amplify an RNA target using a methodology known as nucleic acid sequence based amplification. They are also widely used to transcribe mRNA from a DNA template, a necessary step in protein production.

Much is already known about the RNA polymerase of bacteriophage T7. Bacteriophage T7 RNA polymerase is the product of T7 gene 1; it is a single chain enzyme that requires no auxiliary factors for accurate synthesis in vitro. T7 RNA polymerase alone recognizes its promoters, initiates synthesis, elongates the RNA transcript and terminates synthesis. There is a highly conserved 23 base pair continuous sequence that includes the start site for the initiation of synthesis. In vitro studies have already defined the kinetics of synthesis, the stability of the promoter polymerase complex the contribution of abortive initiation to promoter efficiency and the DNA contacts essential for promoter activity. See U.S. Pat. No. 5,385,834 (Ikeda) and the references cited therein. Limited proteolytic cleavage of T7 RNA polymerase yields a 20 kD amino-terminal fragment and an 80 kD carboxyl terminal fragment. Ikeda, R. A. and Richardson, C. C., J. Biol. Chem. 262: 3790-99 (1987). The carboxyl terminal fragment can initiate RNA synthesis but cannot extend the transcript. Muller, D. K., et al., Biochemistry 28: 3306-13 (1988). Some researchers suggest that the amino terminal domain contains a nonspecific RNA binding site that stabilizes the synthesis complex and permit processive RNA synthesis. Other studies suggest that DNA binding and polymerase activities are distinct functions. Amino acid insertions into the reading frame of the enzyme at residues 640, 648, or 881 inactivate polymerase activity, but promoter binding function is maintained intact. Insertions at residues 159, 222, 240 or 242 disrupt DNA binding, but have no effect on polymerase activity. Gross, L., et al., J. Mol. Biol. 228: 488-505 (1992); Patra, D., et al., J. Mol. Biol. 224: 307-18 (1992). Replacement of Asn748 by Asp, the corresponding residue in T3 RNA polymerase, alters the promoter recognition function. See, Raskin, C. A., et al., J. Mol. Biol. 228: 506-15 (1993).

T7 RNA polymerase recognizes two types of pause/terminations signals. Macdonald, L. E., et al., J. Mol. Biol. 238: 145-58 (1994). Class I signals are typified by the terminator located in the late region of the T7 genome. Such signals encode an RNA that can form a stable stem loop structure followed by an uninterrupted run of U residues. Id.; Dunn, J. J., et al., J. Mol. Biol. 166: 477-535 (1983). Class II signals were first identified in the cloned gene for human preparathyroid hormone (PTH): the signal prevented expression of the gene an a phage RNA polymerase-based system. Mead, D. A., et al., Prot. Eng. 1: 67-74 (1986). Class II signals do not encode an RNA with an apparent secondary structure, but consist of a partially conserved six base pair sequence, H-A-T-C-T-G in which H is C, A, or T, followed by a run of U residues. Macdonald, supra; He, B. et al., J. Biol. Chem. 273: 18802-11 (1998); Lyakhov, D. L. et al., J. Mol. Biol. 280: 201-13 (1998). Class II signals that lack the run of U residues terminate with reduced efficiency but still cause the polymerase to pause. Lyakhov, supra. Such Class II pause sites are found in the concatamer junction of replicating bacteriophage T7 DNA, as well as in bacteriophage T3 and bacteriophage SP6 DNA, and have been shown to cause all of these RNA polymerases to pause/terminate. He, supra; Lyakhov, supra; Zhang, X., et al., J. Mol. Biol. 269: 10-27 (1997).

Because the sequence responsible for class II pause/termination is short it is likely to occur by chance in unrelated DNA. Consequently, when T7 RNA polymerase (or other bacteriophage RNA polymerases such as, for example, T3 and SP6) is used to transcribe DNA, pause/termination can inadvertently prevent expression of the full length gene product in bacteriophage based protein production systems. To circumvent this problem in producing PTH, it was necessary to alter the sequence of the signal in the cloned gene. See Mead, supra. Instead of altering the sequence of every gene having the signal, it would be highly advantageous to use a mutant bacteriophage RNA polymerase in the production system, to eliminate the need to test each gene and modify each of those having the signal sequence.

In addition, on duplex DNA templates that have a protruding 3′ end in the non-template strand, bacteriophage T7 RNA polymerase synthesizes aberrant products due to its ability to insert the exposed end of the non-template strand into the active site and to carry out “tunaround” systhesis in which this strand is then used as a secondary template. Lyakhov, D. L., et al., J. Mol. Biol. 280:201-13 (1998); Schenborn, E. T. and Mierendorf, R. C., Jr., Nuc. Acids Res. 13: 6223-36 (1985); Rong, M., et al., J. Biol. Chem. 273: 10253-60 (1998).

A third problem with using bacteriophage T7 RNA polymerase in synthesis systems is that the enzyme synthesizes far fewer products on templates that terminate in G:C rich sequences compared to templates that terminate in other sequences. See Rong, supra. It is speculated that the greater strength of the RNA:DNA hybrid on G:C rich sequences stabilizes the synthesis complex and slows its release at the end of the template, which lowers the enzyme's turnover rate and inhibits its ability to synthesize as many products as compared to non-G:C rich sequences.

Mutant T7 RNA polymerases that bypass or ignore the class II signal are known. One that has been proteolytically cleaved by the E. coli outer membrane protease ompT, or by trypsin, fails to terminate at class II signals. Macdonald, L. E., et al., J. Mol. Biol. 232: 1030-47 (1993). In addition certain mutant RNA polymerases with alterations in the protease sensitive region near amino acid residues 172-179 fail to terminate at class II signals. Macdonald, L. E., et al., J. Mol. Biol. 238:145-58 (1994); Lyakhov, D. L., et al., J. Mol. Biol. 269: 28-40 (1997). Although not all of the mutations in the region of residues 172-179 confer on the enzyme the ability to bypass the class II signal and continue synthesis of the DNA, the T7 RNA polymerase mutant Δ172-173 in which the two lysine residues at positions 172 and 173 are deleted is an exemplary mutant that does fail to terminate. See Lyakhov, D. L., et al., Molekuliarnaia Biologiia 26: 679087 (1992). Table I provides a list of mutant T7, T3 and SP6 RNA polymerase enzymes, a description of the modifications in the sequence resulting in each mutant and each mutant enzyme's termination/pause efficiency.

Although the ability of the mutant T7 RNA polymerase Δ172-173 to bypass or ignore the class II signal is known, it was unrecognized that this mutant has a number of other as yet unrecognized, apparently novel, properties which make it advantageous for use in the synthesis of homogeneous RNA products.

TABLE I
MUTANT BACTERIOPHAGE RNA POLYMERASES

| ID | GENOTYPE | PLASMID | % T |
|---|---|---|---|
| T7 | | | |
| WT | Wild type | AR1219 | 0.54 |
| WT | Wild type | BH161 | 0.62 |
| K163A | K→A residue at 163 | BH187 | 0.41 |
| K164A | K→A residue at 163 | BH173 | 0.53 |
| ΔK163-4 | Del. K residues at 163 and 164 | B205 | 0.05 |
| ΔK163-9A | Sub. Residues 163-169 → A | BH200 | <0.01 |
| K172G | K → G residue 172 | K172G | 0.53 |
| K172L | K → L residue 172 | K172L | 0.54 |
| K164C | K → C residue 164 | RC11 | ND |
| R173C | R → C residue 173 | RC10 | <0.01 |
| Δ172-3 | Del. K172 and R173 residues | Δ172-3 | <0.01 |
| Δ172-3 | Del. K172 and R173 residues | RC9 | 0.03 |
| 3207 | Sub. Residues 178-193 → WIHM | AR3207 | <0.01 |
| 3239 | Insert G residue at 181 | AR3239 | <0.01 |
| 3280 | Insert G residue at 180 | AR3280 | <0.01 |
| T3 | | | |
| WT | Wild type | CM56 | 0.53 |
| WT | Wild type | DL18 | 0.48 |
| Δ173-4 | Del. K173 and R174 residues | DL60 | <0.01 |
| SP6 | | | |
| WT | Wild type | SR3 | 0.80 |
| WT | Wild type | BH176 | 0.86 |
| Δ140-3 | Del. K140 to R143 residues | AK3 | 0.10 |

ND = not determined

### SUMMARY OF THE INVENTION

Provided are improved materials and methods for reducing the production of aberrant products during RNA synthesis and protein production. Accordingly, in one aspect, the invention includes an improved T7, T3 or SP6 bacteriophage RNA polymerase enzyme, the improved enzyme being characterized by having a significantly diminished ability to displace RNA that causes reduced synthesis of aberrant products on templates having protruding 3′ ends in the non-template strand. In another aspect, the invention includes an improved T7, T3 or SP6 enzyme, the improved enzyme being characterized by the markedly decreased addition of a non-templated nucleotide to the 3′ end of transcripts during the RNA synthesis process. In another aspect, the invention includes an improved bacteriophage T7, T3 or SP6 RNA polymerase enzyme, the improved enzyme being characterized by the ability, during mRNA synthesis from DNA templates, to increase yields of products on DNA templates that terminate in G:C rich sequences to a level comparable to the yields of products on DNA templates that terminate in non G:C rich sequences. Such improved enzymes include bacteriophage T7 RNA polymerase having a deletion of residue number 172 and residue number 173, bacteriophage T3 RNA polymerase having a deletion of residue number 173 and residue number 174, and bacteriophage SP6 RNA polymerase having a deletion of residues 140 through 143.

In another aspect, the invention comprises an improved bacteriophage RNA polymerase enzyme, the improved enzyme having a region of residues present as a disordered loop that does not interact with nucleic acid components in the initiation complex during early stage synthesis. Such improved enzymes include bacteriophage T7 and T3 enzymes having deletions in the region of residues 172-179, and bacteriophage SP6 enzyme having deletions in the region of residues 140-145.

In further aspects, the invention comprises improved methods of synthesizing mRNA from DNA templates. In one such aspect, the invention comprises an improved method of synthesizing homogeneous mRNA from DNA templates comprising transcribing under suitable synthesis conditions DNA templates with a modified bacteriophage RNA polymerase enzyme characterized by having a significantly diminished ability to displace RNA that causes reduced synthesis of aberrant products on templates having protruding 3′ ends in the non-template strand. The improved method may further comprise transcribing under suitable synthesis conditions DNA templates with a modified bacteriophage RNA polymerase enzyme characterized by having a markedly decreased ability to add non-templated nucleotide to the 3′ end of transcripts. The improved method may further include transcribing under suitable synthesis conditions a DNA template with a modified bacteriophage RNA polymerase enzyme characterized by the ability, during mRNA synthesis from DNA templates, to increase yields of products on DNA templates that terminate in G:C rich sequences to a level comparable to the yields of products on DNA templates that terminate in non G:C rich sequences.

Such improved methods comprise transcribing with enzymes including bacteriophage T7 RNA polymerase having a deletion of residue number 172 and residue number 173, bacteriophage T3 RNA polymerase having a deletion of residue number 173 and residue number 174, and bacteriophage SP6 RNA polymerase having a deletion of residues 140 through 143.

In another aspect, the invention comprises an improved method of synthesizing homogeneous mRNA from DNA templates comprising transcribing under suitable synthesis conditions DNA templates with a bacteriophage RNA polymerase enzyme selected from the group consisting of: bacteriophage T7 RNA polymerase having a deletion of residue number 172 and residue number 173, bacteriophage T3 RNA polymerase having a deletion of residue number 173 and residue number 174, and bacteriophage SP6 RNA polymerase having a deletion of residues 140 through 143.

In another such aspect, the invention comprises an improved method of synthesizing homogeneous mRNA from DNA templates comprising transcribing under suitable synthesis conditions DNA templates with a bacteriophage RNA polymerase enzyme characterized by having a region of residues present as a disordered loop that does not interact with nucleic acid components in the initiation complex during early stage synthesis. In this aspect, the improved method of the invention includes transcribing under such suitable synthesis conditions DNA templates with a bacteriophage RNA polymrase enzyme selected from bacteriophage T7 and T3 RNA polymerase enzymes having deletions in the region of residues 172-179, and bacteriophage SP6 enzyme having deletions in the region of residues 140-145. More specifically, the improved method includes transcribing under such suitable synthesis conditions DNA templates with a bacteriophage RNA polymerase enzyme selected from bacteriopage T7 polymerase having a deletion of residues 172 and 173, bacteriophage T3 polymerase enzyme having a deletion of residues 173 and 174, and bacteriophage polymerase enzyme SP6 having a deletion of residues 140 through 143.

The improved method results in reduced synthesis of aberrant products on templates having protruding 3′ ends in the non-template strand, decreased addition of a non-templated nucleotide to the 3′ end of transcripts, and increased yields of products on templates that terminate in G:C rich sequences. The reason the improved method results in these advantages in mRNA synthesis is because modifying the enzyme by deleting the noted residues diminishes the enzyme's ability to displace the RNA which results in the formation of a more extended RNA:DNA hybrid. This decreased ability to displace RNA diminishes the stabilizing interactions of the displaced RNA with the RNA product binding site (the RNA exit pore).

On duplex DNA templates that have a protruding 3′ end in the non-template strand, wild type bacteriophage RNA polymerases synthesize anomalous products due to their ability to insert the exposed end of the non-template strand into the active site and to carry out “turnaround” synthesis in which this strand is then used as a secondary template. The improved enzyme products and methods of the invention exhibit a greatly reduced tendency to carry out this side reaction. The increased dissociation rate of the modified enzyme when it reaches the end of the template decreases the time that the enzyme remains in a position to insert the non-template strand. This explains the enzyme's decreased ability to carry out the undesirable reaction.

On templates that terminate in G:C rich sequences (ie, in which the end of the sequence has a significant number of G and/or C nucleotides), the wild type enzymes synthesize far fewer products than on templates that terminate in other, non G:C rich sequences. It is believed that the greater strength of the RNA:DNA hybrid in such context stabilizes the synthesis complex and thereby slows its release at the end of the template, which in turn lowers the enzyme's turnover rate and results in decreased product yields. The improved enzyme products and methods of the invention exhibit diminished stability of the synthesis complex when it reaches the end of the template. This results in a rate of release and turnover rate, and therefore concomitant product yield, that is comparable to the wild type enzymes on templates that terminate in non G:C rich sequences.

The wild type enzymes are known to add a non-templated nucleotide to the 3′ end of the transcript when it reaches the end of the template. This property is highly undesirable when a homogeneous RNA with a defined 3′ end is required such as when a chemically defined RNA is required. The improved enzyme products and methods of the invention exhibit a markedly decreased tendency to add the non-templated nucleotide due to the enzyme's more rapid dissociation upon reaching the end of the template.

The improved enzymes and methods of the invention include mutations in the sequence of the RNA polymerase enzyme that is involved in displacing the RNA product and resolving the synthesis bubble. During the early stage of synthesis, the RNA polymerase enzyme forms an unstable, initiation complex (IC). After the formation of the IC, the enzyme isomerizes to a stable, highly processive, elongation complex (EC). This isomerization transition from IC to EC is accompanied by a dramatic reorganization and refolding of the N-terminal domain of the enzyme. In the early stage, residues 172-179 of the T7 and T3 enzyme (and 140-145 of the SP6 enzyme) are present as a disordered loop that does not interact with nucleic acid components. During the transition from IC to EC, this loop becomes highly ordered and forms part of a “flap” domain that is involved in stabilizing the RNA:DNA hybrid and in resolving the upstream edge of the synthesis bubble at the location the RNA product is stripped away from the DNA template.

While the improved methods and enzymes of the invention are demonstrated with bacteriophage RNA polymerase enzymes T7, T3 and SP6, it is contemplated that similar modifications may be engineered into other bacteriophage-like RNA polymerase enzymes, such as for example, K11, gh-1 and the mitochrondrial RNA polymerase enzymes, to obtain comparable, improved results in mRNA synthesis and protein production.

The complete nucleotide sequence of bacteriophage T7 DNA and the locations of its genetic elements can be found in Dunn, J. J. and Studier, F. W., J. Mol. Biol. 166: 477-535 (1983). Oligoribonucleotide synthesis using T7, T3, SP6 and K11 RNA polymerase and synthetic DNA templates has been described. See for example, Milligan, J. F., et al., Nuc. Acids Res. 15: 8783-98 (1987); Schenborn, E. T. and Meirendorf, Jr., R. C., Nuc. Acids Res. 13: 622336 (1985) and Zhang, X., and Studier, F. W., J. Mol. Biol. 269: 10-27 (1997). Mutagenesis of bacteriophage RNA polymerases and methods for their purification have been described in He, B, et al., Protein Expression & Purification 9: 142-151 (1997) and in Temiakov, D., t al., Acta Crystallographica, in press (2002). Construction of plasmid templates, and materials and methods used in synthesis and synthesis have been described. See Mentasana, P. E., et al., J. Mol. Biol. 302: 1049-62 (2000) and Gopal, V., et al., J. Mol. Biol. 290: 411-31 (1999). Those skilled in the art will be readily able to construct such other modified RNA polymerase enzymes, test them for the improved properties disclosed herein and employ them in the improved methods of the invention using standard and well known techniques and tools in the art along with the teachings set forth herein. Mutagenesis, production, purification and testing of the mutants for the improved properties disclosed herein can be carried out following the methods described and the publications cited in, inter alia, U.S. Pat. No. 5,385,834 (Ikeda), U.S. Pat. No. 6,531,300B1 (Haydock), U.S. Pat. No. 5,102,802 (McAllister) and U.S. Pat. No. 5,037,745 (McAllister). All publications and patent application cited in this specification are herein incorporated by reference for the substance of their disclosure as if each were specifically and individually indicated to be incorporated by reference.

### FIGURE DESCRIPTIONS

FIG. 1: Illustrates the results discussed in Example 1.

FIG. 2: Illustrates the results discussed in Example 2.

FIG. 3: Illustrates the results discussed in Example 3.

FIG. 4: Illustrates the results discussed in Example 4.

### DETAILED DESCRIPTION

Termination at class II sites requires proper resolution of the synthesis bubble, and is diminished on templates in which this is less likely to occur, for example, on templates in which the non-template strand is missing, or on supercoiled DNA. See, Mead, supra; He, supra; Mentesana, P. E., J. Mol. Biol. 302: 1049-62 (2000); and Hartvig, L. and Christiansen, J., EMBO J. 15: 4767-74 (1996).

We have found that T7 RNA polymerase mutant Δ172-173 exhibits a diminished ability to displace the RNA, resulting in the formation of a more extended RNA:DNA hybrid. Mentesana, P. E., J. Mol. Biol. 302: 1049-62 (2000). This observation explains the failure of this mutant to terminate/pause at class II signals.

The stability of the T7 RNA polymerase elongation complex depends upon a number of nucleic acid:protein interactions. Among these is the association of the displaced RNA with the RNA product binding site, the RNA exit pore. This interaction is of particular importance when the enzyme is halted, for example, as a result of the withholding of a required nucleoside triphosphate (NTP), or at the end of the template. Mentesana, supra; Gopal, V., et al., J. Mol. Biol. 290: 411-31 (1999). Under these conditions, the lack of this interaction results in a more rapid dissociation of the halted complex. The decreased ability of the mutant T7 RNA polymerase to displace the RNA diminishes these stabilizing interactions. This results in a complex that is less stable when slowed or halted. Although this does not affect the properties of the enzyme during elongation under normal conditions, it results in increased dissociation rates when the complex is halted or reaches the end of the template.

On duplex DNA templates that have a protruding 3′ end in the non-template strand, bacteriophage T7 RNA polymerase synthesizes aberrant products due to its ability to insert the exposed end of the non-template strand into the active site and to carry out “tunaround” synthesis in which this strand is then used as a secondary template. We have determined that, in contrast, mutant Δ172-173 T7 RNA polymerase exhibits a greatly reduced tendency to carry out this side reaction. This is because the increased dissociation rate of the modified enzyme when it reaches the end of the template decreases the time that the enzyme remains in a position to insert the non-template strand.

On templates that terminate in a G:C rich sequence, wild type T7 RNA polymerase synthesizes far fewer products than on templates that terminate in other, non-G:C rich, sequences. It is thought that the greater strength of the RNA:DNA G:C rich hybrid stabilizes that synthesis complex and slow its release at the end of the template, resulting in lowering the turnover rate and the concommitant production of transcript. We have determined that, in contrast, mutant Δ172-173 T7 RNA polymerase exhibits a significantly improved rate of synthesis and product yield on G:C rich templates as compared to wild type T7 RNA polymerase. We believe this is due to the diminished stability of the mutant enzyme when it reaches the end of the template. This diminished stability offsets the effect of the greater strength of the RNA:DNA G:C rich hybrid.

Wild type bacteriophage T7 RNA polymerase is also known to add a non-templated nucleotide to the 3′ end of the ttranscript when it reaches the end of the template. Milligan, J. F., et al., Nuc. Acids Res. 15: 8783-98 (1987). This property is highly undesirable in circumstances where homogeneous product with a defined 3′ end is required. We have discovered that the mutant T7 RNA polymerase shows a reduced tendency to carry out this reaction, resulting in higher yields of homogeneous product.

The biochemical basis for the effects of these alterations in enzyme structure can be understood in light of findings that the mutations affect a region of the RNA polymerase that is involved in displacing the RNA product and resolving the synthesis bubble. During the early stage of synthesis the bacteriophage T7 RNA polymerase enzyme forms an unstable initiation complex (IC) before it isomerizes to a stable, highly processive elongation complex (EC). The transition from IC to EC is accompanied by a dramatic reorganization and refolding of the N-terminal domain of the enzyme. In the early stage residues 172-179 are present as a disordered loop that does not interact with nucleic acid components. During the transition from IC to EC, this loop becomes highly ordered and forms part of a “flap” domain that is involved in stabilizing the RNA:DNA hybrid and in resolving the upstream edge of the synthesis bubble at the location the RNA product is stripped away from the DNA template.

The following examples provide additional details of the compositions and processes of the invention. The examples are not intended to restrict the invention, but to illustrate several of the embodiments thereof.

### EXAMPLE 1

### Mutant Bacteriophage T7, T3 and SP6 RNA Polymerases Fail to Terminate at the Class II PHT Signal

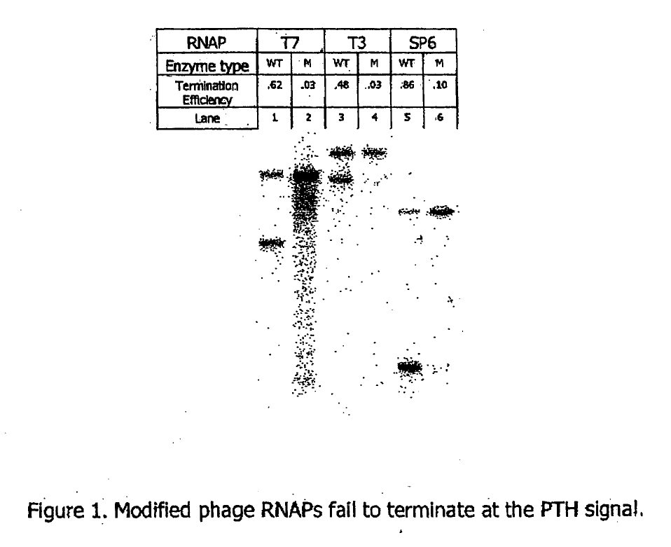

Templates that contain a class II PTH signal downstream from a T7, T3 or SP6 promoter were constructed using well known methods and materials. The T7 templates were transcribed with mutant (M) T7 or wild type (WT) or unmodified T7 enzyme under suitable conditions. The products were resolved by gel electrophoresis. The results are shown in FIG. 1. For each pair of lanes, the upper band represents the runoff, ie, non-terminated product and the lower band represents the terminated product. The efficiency of termination by each enzyme is calculated as:

$$\frac{terminated product}{terminated product + runoff product}$$

The wild type enzymes produced between 48 and 86% terminated product. Wild type T7 produced 62% unwanted terminated product. Wild type T3 produced 48% unwanted terminated product. And wild type SP6 produced 86% unwanted terminated product. In contrast, the mutant enzymes produced significantly less unwanted terminated product: mutant T7 and T3 enzymes produced only 3% and mutant SP6 produced only 6%.

### EXAMPLE 2

### Increased Synthesis of Transcripts on Templates with G:C Rich Ends

Two sets of templates that terminate in two different sequences, the G:C rich sequence (G:C), or the non-G:C rich sequence (N:N), were constructed using known materials and methods. One set of templates was constructed by annealing together synthetic oligomers 59 nucleotides in length that contain a T7 promoter sequence followed by a sequence that directs synthesis of a transcript the ends with either a G:C rich (six G residues) or non-G:C rich (a G residue followed by A and C residues, then a C residue followed by A and C residues) template. The pairs of oligomers used were, respectively, MR98/MR99 for the non-G:C rich template and MR49/MR89 for the G:C rich template. See Rong, J. Biol. Chem 273: 10253-60 (1998). The other set of templates were generated by digestion of pBluescript KS+ (Stratagene) with restriction enzymes EcoRV or SmaI to yield a terminal sequence of either GACTAC in the case of EcoRV or five G residues followed by five C residues in the case of SmaI. The templates were transcribed by wild type (WT) and mutant (M) bacteriophage T7 RNA polymerases under conditions suitable for synthesis. The products were resolved by gel electrophoresis. The synthesized set of template gave rise to a run off transcript of 29 nucleotides that terminated with the sequence GACTAC in the non-G:C rich template and with the sequence G₆:C₆ in the G:C rich template.

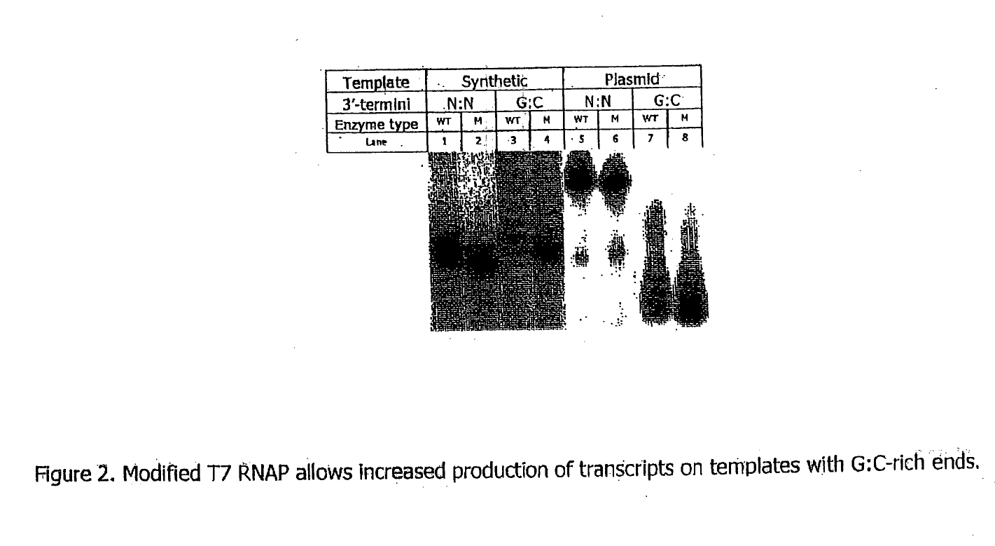

The wild type and mutant forms of the enzyme synthesized nearly equivalent amounts of product on templates that terminate in non G:C rich sequences. On templates that terminate in G:C rich sequences, the mutant form of the enzyme synthesized significantly more product. These results are illustrated in FIG. 2.

### EXAMPLE 3

### Mutant T7 RNA Polymerase Produces RNA with more Homogeneous 3′ Ends

Synthetic templates were constructed using known materials and methods. The templates were designed to give a 29 nucleotide runoff product. Wild type and mutant T7 RNA polymerase enzymes were used to synthesize the templates under suitable synthesis conditions. The synthesis products were resolved by gel electrophoresis in a 20% gel.

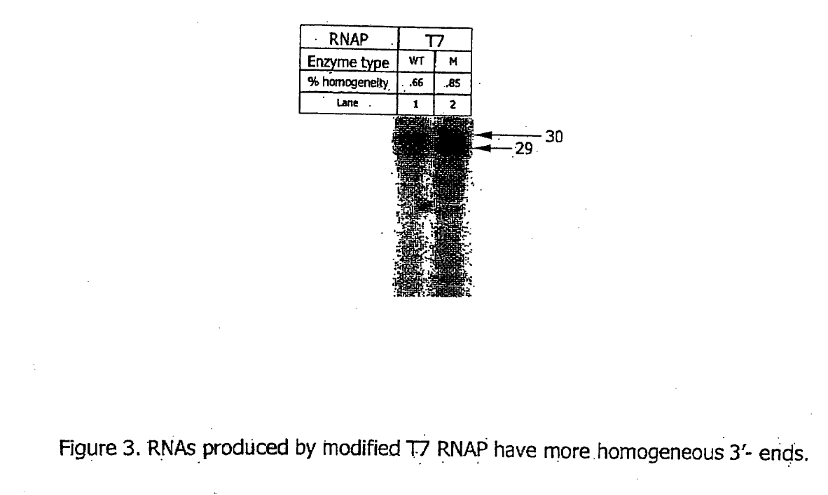

The results are illustrated in FIG. 3. In addition to the expected 29 nucleotide runoff product (n), the wild type enzyme (WT) produced a 30 nucleotide transcript due to the addition of a non-templated nucleotide to the 3′ end (n+1, lane 1). The mutant enzyme (M) produced a higher percentage of product of the expected size of 29 nucleotides (lane 2). The homogeneity of the product (as % homogeneity) is calculated as the percent of correctly terminated product versus total product and shown in the figure.

### EXAMPLE 4

### Use of Mutant T7 RNA Polymerase Results in Decreased Synthesis of Aberrant Product on Templates with Protruding 3′ Ends

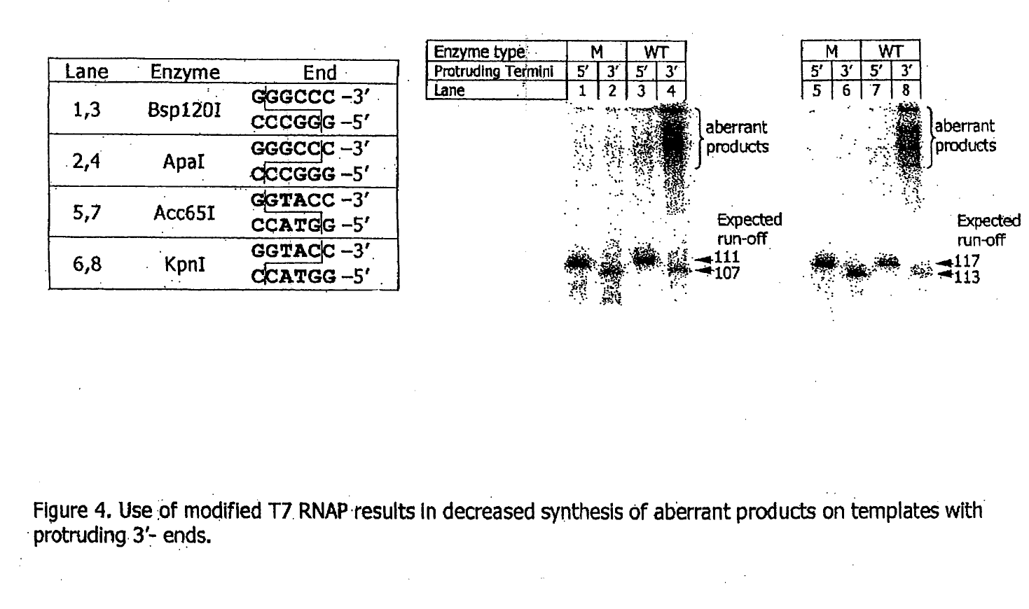

The plasmid pBluescript IIKS+ (Stratagene) was digested with Bsp1201 or with ApaI using known materials and methods. Bsp1201 and ApaI recognize the sequence GGGCCC. Digestion with Bsp1201 generates a sequence with a 5′ protruding end and digestion with ApaI generates a sequence with a 3′ protruding end. Additional plasmid was digested with Acc651 or KpnI under the same experimental conditions. Acc651 and KpnI recognize the sequence GGTACC. Digestion with Acc651 generates a sequence with a 5′ protruding end and digestion with KpnI generates a sequence with a 3′ protruding end. See FIG. 4A.

The digested templates were transcribed under suitable synthesis conditions with wild type bacteriophage T7 RNA polymerase enzyme (WT) and with mutant bacteriophage T7 RNA polymerase enzyme (M) and the products resolved by polyacrylamide gel electrophoresis (6%) in a manner well known in the art. The results are illustrated in FIG. 4. Each panel shows the positions of the expected runoff products and the position of products that would arise from aberrant synthesis by turnaround synthesis on templates having protruding 3′ ends.

The above detailed description is for illustrative purposes and is not intended to limit the spirit or the scope of the invention or its equivalents as defined in the appended claims.

We claim:

1. An improved bacteriophage RNA polymerase enzyme, the improved enzyme being characterized by having a significantly diminished ability to displace RNA that causes reduced synthesis of aberrant products on templates having protruding 3′ ends in the non-template strand.

2. The improved enzyme according to claim 1, being further characterized by the markedly decreased addition of a non-templated nucleotide to the 3′ end of transcripts during the RNA synthesis process.

3. The improved enzyme according to claim 2, being additionally characterized by the ability, during mRNA synthesis from DNA templates, to increased yields of products on templates that terminate in G:C rich sequences to a level comparable to the yields of products ion DNA templates that terminate in non G:C rich sequences.

4. The improved enzyme according to claim 3, being additionally characterized by having a region of residues present as a disordered loop that does not interact with nucleic acid components in the initiation complex during early stage synthesis.

5. The improved enzyme according to claim 4 wherein said enzyme is bacteriophage T7 RNA polymerase having a deletion of residue number 172 and residue number 173.

6. An improved enzyme according to claim 4 wherein said enzyme is bacteriophage T3 RNA polymerase having a deletion of residue number 173 and residue number 174.

7. An improved enzyme according to claim 4 wherein said enzyme is bacteriophage SP6 RNA polymerase having a deletion of residues 140 through 143.

8. An improved method of synthesizing homogeneous mRNA from DNA templates comprising transcribing under suitable synthesis conditions DNA templates with a modified bacteriophage RNA polymerase enzyme characterized by having a significantly diminished ability to displace RNA that causes reduced synthesis of aberrant products on templates having protruding 3′ ends in the non-template strand.

9. An improved method according to claim 8 wherein said enzyme is additionally characterized by having a markedly decreased ability to add non-templated nucleotide to the 3′ end of transcripts.

10. An improved method according to claim 9 wherein said enzyme is additionally characterized by having the ability, during mRNA synthesis from DNA templates, to increase yields of products on DNA templates that terminate in G:C rich sequences to a level comparable to the yields of products on DNA templates that terminate in non G:C rich sequences.

11. An improved method according to claim 10 wherein said enzyme is additionally characterized by having a region of residues present as a disordered loop that does not interact with nucleic acid components in the initiation complex during early stage synthesis.

12. The improved method according to claim 11 wherein said enzyme is selected from bacteriophage T7 and T3 RNA polymerase enzymes having deletions in the region of residues 172 to 179 and from bacteriophage SP6 RNA polymerase enzyme having deletions in the region of residues 140 to 145.

13. The improved method according to claim 12 wherein said bacteriophage T7 RNA polymerase enzyme has a deletion of residues 172 and 173.

14. The improved method according to claim 12 wherein said bacteriophage T3 RNA polymerase enzyme has a deletion of residues 173 and 174.

15. The improved method according to claim 12 wherein said bacteriophage SP6 RNA polymerase enzyme has a deletion of residues 140 through 143.
